(19) United States
(12) Patent Application Publication
(10) Pub. No.: US 2026/0162269 A1
(43) Pub. Date: Jun. 11, 2026

(54) BURN INJURY ASSESSMENT METHOD AND SYSTEM

(71) Applicant: NATIONAL CHENG KUNG UNIVERSITY, Tainan (TW)

(72) Inventors: CHIH-LUNG LIN, Tainan (TW); YU-HSIEN LU, Kaohsiung (TW); MENG-HSUAN WU, Tainan (TW)

(21) Appl. No.: 19/409,726

(22) Filed: Dec. 4, 2025

Related U.S. Application Data
(60) Provisional application No. 63/728,254, filed on Dec. 5, 2024.

(51) Int. Cl. G06T 7/00; A61B 5/00; A61B 5/01; A61B 5/1455; G06T 7/11; G06T 7/521

(52) U.S. Cl. CPC G06T 7/0016; A61B 5/0077; A61B 5/015; A61B 5/14551; A61B 5/443; A61B 5/445; G06T 7/11; G06T 7/521; A61B 2560/0228; G06T 2207/10028; G06T 2207/10048; G06T 2207/20081; G06T 2207/20084; G06T 2207/30088

(57) ABSTRACT

A burn injury assessment method is provided. The method includes: acquiring a visible light image of a wound, a depth image of the wound, a multispectral image of the wound, a thermal image of the wound, and an injury time parameter; inputting the visible light image into an image segmentation model to obtain a wound region and a wound region mask; aligning the depth image, the multispectral image, the visible light image, and the thermal image to generate an aligned image; calculating a plurality of physiological parameters of the wound region and a wound region area of the wound region based on the wound region mask and the aligned image; and inputting the wound region area, the plurality of physiological parameters, and the injury time parameter into a burn classification model to obtain a burn index of the wound.

### CROSS-REFERENCE TO RELATED APPLICATION(S)

The present application claims the benefit of and priority to U.S. Provisional Patent Application Ser. No. 63/728,254, filed on Dec. 5, 2024, entitled “BURN INJURY CLASSIFICATION AND CARE RECOMMENDATION SYSTEM”, the contents of which are hereby incorporated herein fully by reference into the present application for all purposes.

### FIELD

The present disclosure generally relates to a wound assessment method and system, and more particularly, to a burn injury assessment method and system.

### BACKGROUND

In clinical practice, wound surface area assessment is commonly performed using the rule of nines, the Lund and Browder chart, or the palm method. These techniques, however, rely heavily on a subjective estimation by a physician. In addition, body proportion differences among individuals could lead to inaccuracies, thus preventing precise quantification of wound areas. Conventionally, degree of burns could be classified by analyzing color images of the wound and extracting features from the wound, deep tissue information could be obtained by analyzing hyperspectral imaging (HSI) of the wound to capture hundreds of spectral bands, and the temperature of the wound could be obtained by analyzing infrared thermal images of the wound.

However, color images can only collect information from the burnt surface. With hyperspectral imaging, as the number of collected spectral bands increases, the cost and data complexity also increase. As for infrared thermal imaging, variations in environmental temperature and observation angle can also lead to erroneous classification results. Furthermore, regarding the assessment of the degree of burns, it is necessary to analyze the degree of burns, through a physician's visual judgment of the wound surface condition, three days after the burn occurs. Such analysis not only requires more time for diagnosis but also prevents timely assessment of the wound when the burn occurs. Such analysis may also cause diagnostic errors due to the subjective opinions of different physicians, thus potentially leading to delays in treatment.

### SUMMARY

In view of the above, there is a need for a burn injury assessment system and method to assist in clinical judgment, thus enabling medical personnel to evaluate burn wounds based on recommendations, thereby supporting comprehensive treatment and follow-up for the burn wounds.

The present disclosure provides a burn injury assessment method, as well as a burn injury assessment system configured to implement the method. By using the burn injury assessment method, recommendations, such as an individual's wound area and a burn index, could be effectively provided based on a target wound image, and the recommendations facilitate assisting medical personnel in planning subsequent medical treatments in advance. The present disclosure could more accurately and timely analyze information of a burn injury to facilitate early assessment of the degree of burns, thus allowing a physician to provide earlier treatment based on the wound's condition. Currently known degree of burns are indicators used in hospitals to classify the severity of burns, which is divided into first degree through fourth degree only differentiated based on a burn depth. However, the burn index is an indicator proposed by the present disclosure. In addition to the conventional degree of burns, the burn index further incorporates a burn area, an injury time parameter, the blood oxygen content of the wound, and the wound temperature, thereby enabling a more comprehensive assessment of the severity of the burn and the degree of recovery. In the burn index, a higher score represents a more severe burn, while a lower score represents a milder burn. Furthermore, during the recovery process, the degree of burn recovery could be better evaluated by continuously tracking the burn index using the burn analysis system.

According to a first aspect of the present disclosure, a burn injury assessment method is provided. The method includes acquiring a visible light image of a wound, a depth image of the wound, a multispectral image of the wound, a thermal image of the wound, and an injury time parameter; inputting the visible light image into an image segmentation model to obtain a wound region and a wound region mask; aligning the depth image, the multispectral image, the visible light image, and the thermal image to generate an aligned image; calculating a plurality of physiological parameters of the wound region and a wound region area of the wound region based on the wound region mask and the aligned image; and inputting the wound region area, the plurality of physiological parameters, and the injury time parameter into a burn classification model to obtain a burn index of the wound.

In an implementation of the first aspect of the present disclosure, a burn injury assessment method is provided. The method includes acquiring a visible light image of a wound, a depth image of the wound, one or more multispectral images of the wound, a thermal image of the wound, and an injury time parameter; inputting the visible light image into an image segmentation model to obtain a wound region and a wound region mask; aligning the depth image, the one or more multispectral images, the visible light image, and the thermal image to generate an aligned image; calculating a plurality of physiological parameters of the wound region and a wound region area of the wound region based on the wound region mask and the aligned image; and inputting the wound region area, the plurality of physiological parameters, and the injury time parameter into a burn classification model to obtain a burn index of the wound.

In an implementation of the first aspect of the present disclosure, the image segmentation model comprises a fully convolutional network (FCN).

In another implementation of the first aspect of the present disclosure, the burn classification model comprises a multilayer perceptron (MLP).

In another implementation of the first aspect of the present disclosure, the plurality of physiological parameters comprise a wound depth, a water content, a blood oxygen concentration value, and a temperature of the wound.

In another implementation of the first aspect of the present disclosure, acquiring the multispectral image of the wound includes using at least one green light source, at least one red light source, and at least one near-infrared light source.

In another implementation of the first aspect of the present disclosure, a wavelength of the at least one green light source is between 510 nm and 560 nm, a wavelength of the at least one red light source is between 630 nm and 680 nm, and a wavelength of the at least one near-infrared light source is between 730 nm and 950 nm.

In another implementation of the first aspect of the present disclosure, the method further includes inputting the thermal image into a temperature calibration model to obtain a calibrated thermal image.

In another implementation of the first aspect of the present disclosure, the method further includes calculating a wound healing score based on the wound region area, the plurality of physiological parameters, and the burn index.

In another implementation of the first aspect of the present disclosure, the method further includes calculating a plurality of depth values of the wound region in the depth image based on the wound region mask and the depth image; projecting the plurality of depth values onto a three-dimensional human-body model to obtain three-dimensional information; and converting the three-dimensional information into a two-dimensional image to calculate a ratio of the wound region area relative to a total skin surface area.

According to a second aspect of the present disclosure, a burn injury assessment system is provided. The burn injury assessment system including at least one processor, and at least one memory coupled to the at least one processor and storing at least one computer-executable instruction that, when executed by the at least one processor, causes the burn injury assessment system to execute the burn injury assessment method of the first aspect of the present disclosure.

### BRIEF DESCRIPTION OF THE DRAWINGS

The present patent or application file contains at least one drawing executed in color. Copies of the present patent or patent application publication with color drawing(s) will be provided by the Office upon request and payment of the necessary fee. The present disclosure will be better understood from the following detailed description read in light of the accompanying drawings, where:

FIG. 1 is a schematic block diagram illustrating a burn injury assessment system according to an example implementation of the present disclosure.

FIG. 2 is a schematic diagram illustrating an image capturing device and a wound according to an example implementation of the present disclosure.

FIG. 3 is a flowchart of a burn injury assessment method according to an example implementation of the present disclosure.

FIG. 4 is a schematic diagram of image segmentation according to an implementation of the present disclosure.

### DETAILED DESCRIPTION

The detailed description provided below in connection with the appended drawings is intended as a description of the present examples and is not intended to represent the only forms in which the present examples may be constructed or utilized. The description sets forth the functions of the examples and the sequence of steps for constructing and operating the examples. However, the same or equivalent functions and sequences may be accomplished by different examples.

For convenience, certain terms employed in the specification, examples, and appended claims are collected here. Unless otherwise defined herein, scientific, and technical terminologies employed in the present disclosure shall have the meanings that are commonly understood and used by one of ordinary skill in the art. Also, unless otherwise required by context, it will be understood that singular terms shall include plural forms of the same, and plural terms shall include the singular. Specifically, as used herein and in the claims, the singular forms “a” and “an” include the plural reference unless the context clearly indicates otherwise. Also, as used herein and in the claims, the terms “at least one” and “one or more” have the same meaning and include one, two, three, or more.

Terms, such as “at least one embodiment”, “one embodiment”, “multiple embodiments”, “different embodiments”, “some embodiments”, “present embodiment”, and the like may indicate that an embodiment of the present disclosure so described may include a particular feature, structure, or characteristic, but not every possible embodiment of the present disclosure must include a particular feature, structure, or characteristic. Furthermore, repeated use of the phrases “in one embodiment”, “in the embodiment”, and so on does not necessarily refer to the same embodiment, although they may be identical. Furthermore, the use of phrases, such as “embodiments” in connection with “the present disclosure” does not imply that all embodiments of the present disclosure necessarily include a particular feature, structure, or characteristic, and should be understood as “at least some embodiments of the present disclosure” include the particular feature, structure, or characteristic described.

Additionally, for the purposes of explanation and non-limitation, specific details, such as functional entities, techniques, protocols, standards, and the like, are set forth for providing an understanding of the described technology. In other examples, detailed disclosure of well-known methods, technologies, systems, architectures, and the like are omitted so as not to obscure the disclosure with unnecessary details.

The terms “first”, “second”, and “third” in the description of the present disclosure and the above-mentioned drawings are used to distinguish different objects, rather than to describe a specific order.

Furthermore, the term “comprising” and any variations thereof are intended to cover non-exclusive inclusions and may refer to “including but not necessarily limited to”, which specifically indicates open-ended inclusion or membership in the so-described combination, group, series, and the equivalent. For example, a process, method, system, product, or device that includes a series of steps or modules is not limited to the listed steps or modules, but optionally also includes steps or modules that are not listed, or optionally also includes other steps or modules that are inherent to those processes, methods, products, or devices.

The implementations of the present disclosure are described below with reference to the accompanying drawings.

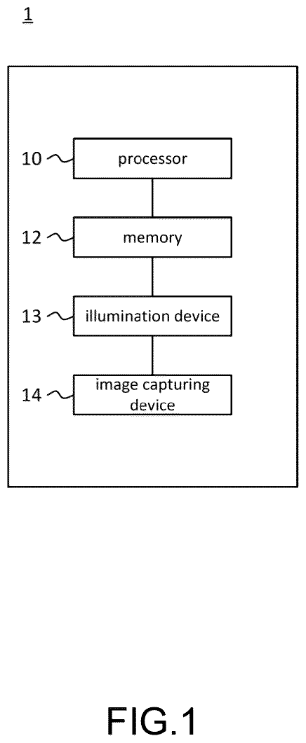

FIG. 1 is a schematic block diagram illustrating a burn injury assessment system according to an example implementation of the present disclosure.

In some implementations, a burn injury assessment system 1 includes a processor 10, a memory 12, an illumination device 13, and an image capturing device 14.

In some implementations, the processor 10 is responsible for running the main computation process and related control logic for algorithms, such as deep learning. The processor 10 may be implemented through a central processing unit (CPU), or may be implemented programmable general-purpose or special-purpose microprocessors, digital signal processors (DSPs), programmable controllers, application-specific integrated circuits (ASICs), programmable logic devices (PLDs), or other similar components or combinations of these components.

In some implementations, the burn injury assessment system 1 may include a memory 12 that stores computer-executable instructions. The memory 12 may include any form of fixed or removable random access memory (RAM), read-only memory (ROM), flash memory, hard disk, or other similar components, or combinations of these components. The memory is used to store executable instructions that enable the processor 10 to execute the instructions, thus allowing the burn injury assessment system 1 to perform the various operations described in the present disclosure.

In some implementations, the burn injury assessment system 1 may further include an illumination device 13 and an image capturing device 14. The illumination device 13 may include any suitable one or more illumination units configured to provide visible light, red light, green light, or near-infrared light. In some implementations, the red light may have a wavelength between 630 and 680 nanometers (nm), the green light may have a wavelength between 510 and 560 nm, and the near-infrared light may have a wavelength between 730 and 950 nm. However, the present disclosure is not limited to any specific wavelength ranges for the visible, red, green, or near-infrared light.

In some implementations, the image capturing device 14 is configured to capture a target image, and may be any imaging device suitable for capturing such target images.

In some implementations, the image capturing device 14 may include a charge-coupled device (CCD) sensor, a complementary metal-oxide semiconductor (CMOS) sensor, or any other appropriate photosensitive component. In some implementations, the image capturing device 14 may also include a thermal imaging sensor. In some implementations, the image capturing device 14 may further include a device capable of sensing scene depth, such as a depth sensing camera. In some implementations, the image capturing device 14 may include a device capable of sensing different frequency ranges of light sources, such as a multispectral camera, to obtain multispectral images. The acquired multispectral images may include red light images, green light images, visible light images, and near-infrared light images.

In some implementations, the burn injury assessment system 1 may further include a display device (not shown in the figure) that may be implemented using a liquid crystal display (LCD), a light-emitting diode (LED) display, a field emission display (FED), or other types of displays, to output various information to the user. In some implementations, the display device may include a speaker, a communication interface, or a combination of these components. In other words, the present disclosure does not limit the form of the output, and one skilled in the art may design the display device to output various types of information according to their needs.

In some implementations, the burn injury assessment system 1 may further include a wireless communication unit (not shown in the figures). In some implementations, the processor 10 may obtain an injury time parameter of an individual from a cloud database via the wireless communication unit. In some implementations, the processor 10 may transmit a burn index that is obtained from an analysis of the burn injury image, together with wound recovery information, to the cloud database through the wireless communication unit.

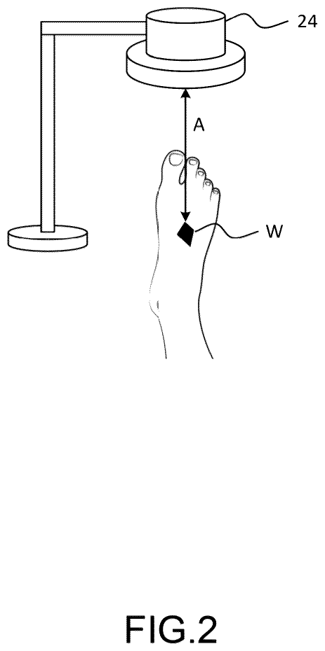

FIG. 2 is a schematic diagram illustrating an image capturing device and a wound according to an example implementation of the present disclosure.

Referring to FIG. 2. In some implementations, the burn injury assessment system includes the image capturing device 24. There is a distance A between the image capturing device 24 and the wound W, where the distance A is between 13 cm to 17 cm.

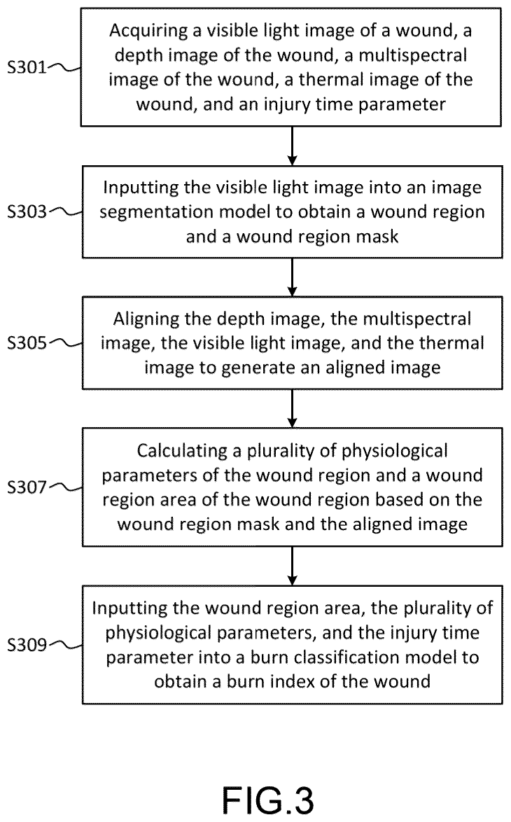

FIG. 3 is a flowchart of a burn injury assessment method according to an example implementation of the present disclosure. The burn injury assessment method may be executed by the burn injury assessment system.

Please refer to FIG. 3, in step S301, acquiring a visible light image of a wound, a depth image of the wound, a multispectral image of the wound, a thermal image of the wound, and an injury time parameter. The visible light image of a wound, the depth image of the wound, the multispectral image of the wound, the thermal image of the wound, and the injury time parameter may be provided by the burn injury assessment system. The injury time parameter may represent a time interval, measured in hours, between occurrence of the injury and the capturing of the wound images, and the injury time parameter may be supplied by the cloud database. In some implementations, the burn injury assessment system may capture images of an individual's wound to obtain the visible light image, the depth image, the multispectral image, and the thermal image. The visible light image, the depth image, the multispectral image, and the thermal image may then be analyzed by the processor 10.

To determine the condition of a burn injury, characteristics of the burn injury, such as wound depth, blood oxygen concentration, and tissue water content may be analyzed to evaluate the extent of damage to the skin or subcutaneous tissues. Specifically, following the occurrence of a burn injury, the skin and subcutaneous tissues undergo a series of dynamic physiological changes over time, which could generally be divided into three major phases. In the acute phase (within approximately 0 to 24 hours after injury), tissue sustains direct thermal damage, and the vasculature initially experiences transient constriction followed by rapid dilation due to the release of inflammatory mediators. The above physiological condition in acute phase will result in unstable local blood flow, a marked decrease in blood oxygen concentration (e.g., StO₂), reduced tissue water content caused by plasma extravasation, and loss of extracellular fluid. During the subacute phase (approximately 1 to 3 days after injury), inflammatory responses peak, capillary permeability increases substantially, and exudate accumulation leads to pronounced local edema, thereby increasing tissue water content. However, microcirculatory function remains partially impaired, hemoglobin remains in a deoxygenated state, and blood oxygen concentration persists at a reduced level. When the wound enters the repair phase (beyond approximately 3 days after injury), neovascularization gradually develops, local circulatory function begins to recover, blood oxygen concentration increases, and tissue water content stabilizes as edema subsides and tissue remodeling progresses. These phase-dependent patterns of physiological parameter changes indicate that injury time is a key factor in determining the current physiological status of a wound.

The severity level of a burn injury also significantly influences the physiological parameter profile of the affected tissues. First degree burns are limited mainly to the epidermis, with microvascular structures remaining largely intact. Therefore, local blood oxygen concentration and tissue water content remain near normal, and the skin appears erythematous with preserved pain sensation. Second degree burns extend into the dermis and result in partial microvascular damage, thus causing reduced local blood flow, noticeably decreased blood oxygen concentration, and elevated tissue water content due to inflammatory edema. Exudation and blister formation are commonly observed. Third degree burns involve full-thickness destruction of skin tissue, including the epidermis, dermis, and subcutaneous layers. Microvascular function is lost, local blood oxygen concentration becomes extremely low, and tissue water content decreases markedly due to tissue necrosis and water evaporation, thus producing a dry, pale, or charred appearance. Fourth degree burns extend beyond the full-thickness skin layers into subcutaneous tissue, muscle, and bone, resulting in necrosis of adipose tissue, muscle, nerves, and bone, with a carbonized presentation. Accordingly, the degree of burns dictates the degree of microcirculatory impairment and the magnitude and direction of tissue water-content changes, thus serving as a major determinant of the absolute values of physiological parameters.

In clinical practice, the time at which a patient seeks medical attention is highly variable, and burn injuries of identical severity may exhibit distinct physiological parameter combinations at different post-injury time points. For example, for a second degree burn injury, if the patient presents for treatment 0.5 hours after injury, the measured blood oxygen concentration may be moderately reduced and tissue water content slightly decreased. However, if treatment is sought 48 hours after injury, the measured blood oxygen concentration may be similar or even lower, but tissue water content may be significantly increased due to peak edema. Thus, relying solely on single-time-point values of blood oxygen concentration and tissue water content may lead to misinterpretation. Accordingly, the present disclosure simultaneously measures multiple physiological parameters, including injury time parameter, wound depth, blood oxygen concentration, and tissue water content to establish a burn classification model that correlates physiological parameters with injury time. The injury time parameter serves as a calibration factor for adjusting the interpretation of the other physiological indicators. In some implementations, the injury time parameter represents the time interval, measured in hours, from injury occurrence to clinical presentation. In some implementations, the injury time parameter represents the time interval, measured in hours, from the injury occurrence to image acquisition after the clinical presentation. The burn injury assessment method enables a more comprehensive and accurate determination of the wound's physiological stage and the extent of microcirculatory damage, thereby allowing estimation of the actual depth of subcutaneous tissue injury and significantly improving the accuracy of the burn index evaluation.

In some implementations, if a burn injury assessment system detects a combination of low blood oxygen concentration and low tissue water content without considering time related factors, such a combination may correspond to a second degree burn during the acute phase (e.g., approximately 2 hours after injury). However, an identical combination of a low oxygen saturation and a low water content may also correspond to a third degree burn during the subacute phase (e.g., approximately 48 hours after injury). By incorporating the injury time parameter, the present disclosure provides the burn classification model with essential temporal context, thus enabling accurate differentiation between different clinically distinct burn classifications. Consequently, the disclosed approach allows reliable and accurate assessment of burn severity.

In some implementations, the processor 10 may analyze the multispectral image to obtain physiological parameters of the wound, such as wound depth, blood oxygen concentration, and tissue water content. Specifically, the image capturing device 14 may capture the wound using at least one green light source, at least one red light source, and at least one near-infrared light source to obtain the multispectral image of the wound. The green light source may have a wavelength between 510 nm and 560 nm, the red light source may have a wavelength between 630 nm and 680 nm, and the near-infrared light source may have a wavelength between 730 nm and 950 nm.

For example, the wavelength of the green light source may be 530 nm, the wavelength of the red light source may be 660 nm, and the wavelengths of the near-infrared light sources may be 730 nm and 880 nm. The processor 10 may analyze the multispectral images obtained at the foregoing wavelengths based on the Beer-Lambert law to determine physiological parameters of the wound, such as blood oxygen concentration and tissue water content.

In some implementations, a multispectral image, obtained by using the near-infrared light sources with the wavelengths of 810 nm and 940 nm, and a vertical-cavity surface-emitting laser (VCSEL) having a wavelength of 850 nm, may be further utilized to enhance the penetration depth of the light into the skin. The processor 10 may analyze the multispectral images based on the Beer-Lambert law to determine the wound depth.

In some implementations, the image capturing device 14 may include a thermal imaging sensor (for example: Optris Xi400), and the thermal imaging sensor may be used to acquire a thermal image of the wound region. The processor 10 may then analyze the thermal image based on a radiometric conversion formula to determine the temperature of the wound, where the radiometric conversion formula may be provided by the manufacturer of the thermal imaging sensor.

In some implementations, a depth sensing camera included in the image capturing device 14 may be used to acquire a visible light image and a depth image of the wound region. The processor 10 may then analyze the visible light image and the depth image to obtain the wound area.

In some implementations, the visible light image, depth image, multispectral image, and thermal image may be provided by a burn injury image database.

Referring to FIG. 3, in step S303, inputting the visible light image into an image segmentation model to obtain a wound region and a wound region mask.

In some implementations, after the visible light image is input into the image segmentation model, a wound region and a non-wound region may be obtained. The image segmentation model may include a Fully Convolutional Network (FCN), a DeeplabV3+ model, a LinkNet model, or a Feature Pyramid Network (FPN) model. In some implementations, the image segmentation model is preferably a Fully Convolutional Network (FCN). In some implementations, the image segmentation model may be trained by labeling wound regions or non-wound regions in the visible light image.

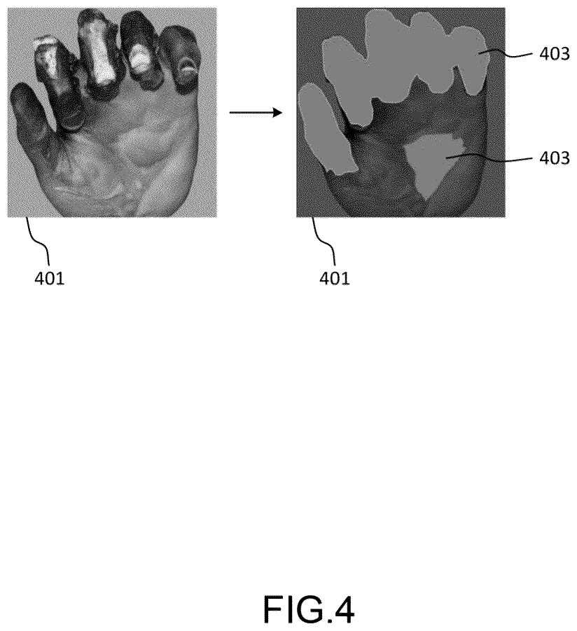

FIG. 4 is a schematic diagram of image segmentation according to an implementation of the present disclosure.

For example, referring to FIG. 4, by analyzing the visible light image 401 of the wound using the image segmentation model, a wound region 403 and a non-wound region in the visible-light image 401 may be output, where the non-wound region corresponds to portions of the visible light image 401 that do not belong to the wound region 403. Specifically, the wound region 403 may be regarded as a wound image mask configured to indicate the location of the wound. The foregoing segmentation of the visible light image may alternatively be performed using any type of image processing algorithm, and the present disclosure is not limited thereto.

Please referring to FIG. 3, in step S305, aligning the depth image, the multispectral image, the visible light image, and the thermal image to generate an aligned image.

In some implementations, an image alignment procedure is performed by the burn injury assessment system to align the depth image, the multispectral image, the visible light image, and the thermal image. As used herein, the terms “image registration” or “image alignment” refer to a process of transforming multiple datasets into a common coordinate system. Within the scope of the present disclosure, image alignment is applied to multiple medical images. Specifically, one image is designated as a target image, and the other images are designated as source images or moving images. The image alignment procedure involves spatially transforming the source images so as to align them with the target image.

In some implementations, for example, execution of the image alignment procedure by the burn injury assessment system may begin by detecting multiple features in the depth image, the multispectral image, the visible light image, and the thermal image. Specifically, algorithms, such as Scale-Invariant Feature Transform (SIFT), edge-detection algorithms, or corner-detection algorithms, may be used for this purpose. In some implementations, one or more markers may be disposed on the individual, and the burn injury assessment system may identify such markers in the depth image, the multispectral image, the visible light image, and the thermal image. The features or markers identified across the multiple images may then be matched, and a homography matrix may be computed accordingly. The coordinates of one image may subsequently be transformed to the coordinate system of another image based on the computed homography matrix. It should be understood that any conventional algorithm in the field of computer vision capable of performing image alignment may be employed, and the present disclosure is not limited herein.

In some implementations, the thermal images merely reflect thermal radiation intensity and do not allow precise identification of wound location, the thermal image is aligned with the visible light image such that each pixel in the thermal image corresponds to a corresponding position in the visible light image. Aligning the thermal image with the visible light image enables accurate computation of temperatures within the wound region or the non-wound region in subsequent analysis.

Referring again to FIG. 3, in step S307, calculating a plurality of physiological parameters of the wound region and a wound region area of the wound region based on the wound region mask and the aligned image.

In some implementations, as described in step S303, the wound region mask is obtained after the visible light image is input into the image segmentation model. The burn injury assessment system may then compute multiple physiological parameters and the area of the wound region based on the wound region mask and the aligned images. The physiological parameters include wound depth, water content, blood oxygen concentration values, and temperature of the wound.

Because the sizes of the different types of images may not be identical, applying the wound mask separately to each individual image in order to analyze wound region of each image would require independently delineating the wound location for each image, which can introduce analytical errors and significantly increase processing time. However, once the depth image, multispectral image, visible light image, and thermal image are aligned, an aligned image having a unified image dimension is generated, with all pixels of the depth image, multispectral image, visible light, image and thermal image mutually aligned. Accordingly, delineation of the wound region in only one of the aligned depth image, multispectral image, visible light image, or thermal image may be applied uniformly to the others.

In some implementations, the processor 10 may analyze physiological parameters of the wound region in the multispectral image based on the wound region mask and the multispectral image of the aligned images. Such analysis is based on the optical characteristics of specific biomolecules (e.g., various forms of hemoglobin and water molecules), which selectively absorb light of different wavelengths. Specifically, the processor 10 may perform computations on multispectral image acquired under specific wavelength bands, such as green light (e.g., wavelengths between 510 nm and 560 nm, particularly about 530 nm), red light (e.g., wavelengths between 630 nm and 680 nm, particularly about 660 nm), and near-infrared light (e.g., wavelengths between 730 nm and 950 nm, particularly about 730 nm and 880 nm). Based on the Beer-Lambert Law, the processor 10 may calculate blood oxygen concentration values and water content of the wound region, where the blood oxygen concentration values include oxyhemoglobin (HbO₂), tissue oxygen saturation (StO₂), and deoxygenated hemoglobin (Hb).

In some implementations, the processor 10 may also determine wound depth for the wound region based on the wound region mask and the multispectral image. To achieve depth measurement, the image capturing device 24 may employ a specially configured combination of light sources designed to maximize light penetration into skin tissue. For example, the light source combination may include near-infrared light sources having wavelengths of approximately 810 nm and 940 nm, and may further employ a vertical cavity surface emitting laser (VCSEL) having a wavelength of approximately 850 nm. These specific wavelengths, particularly the VCSEL with high collimation, could effectively penetrate superficial tissue layers and reflect from deeper structures. Accordingly, the processor 10 may analyze image features acquired under the above light source configuration (e.g., light intensity attenuation or changes in spot pattern characteristics) and determine the depth information for the wound region based on the Beer-Lambert Law.

In some implementations, the processor 10 may calculate multiple depth values corresponding to the wound region within the depth image based on the wound region mask and the depth image, and may project the multiple depth values onto a human three-dimensional model to obtain three-dimensional information. The three-dimensional information is then converted into a two-dimensional image to compute the ratio of the wound region area relative to the total skin surface area of the individual.

In some implementations, the depth sensing camera may capture both a visible light image and a depth image of the wound region. The depth image may be processed into a set of raw point cloud data representing three-dimensional spatial coordinates of the wound and adjacent tissue. The processor 10 may then compute the depth values of the pixels within the wound region mask based on the corresponding depth image.

The processor 10 may subsequently project the pixels containing depth information (i.e., the three-dimensional point cloud) onto a predefined or customized three-dimensional human-body model, thereby constructing three-dimensional geometric information of the wound region (e.g., a 3D mesh model). Based on the three-dimensional geometric information, the processor 10 may accurately compute the actual three-dimensional surface area of the wound, taking into account the curvature and contour of the wound surface. Such approach provides a more accurate evaluation of wound size compared to traditional two-dimensional planar computations.

Then, to improve the accuracy and efficiency of subsequent three-dimensional reconstruction, the processor 10 may perform one or more preprocessing operations on the raw point cloud data. The preprocessing operations are intended to remove noise, eliminate outlier points, and smooth the data. For example, the preprocessing operations may include applying a statistical outlier removal filter or a DBSCAN-based outlier filter to eliminate invalid measurement points; performing voxel-grid downsampling to reduce data density; and/or applying a Gaussian smoothing algorithm or a bilateral smoothing algorithm to produce a smoother point cloud surface.

After preprocessing, the processor 10 may convert the optimized point cloud data into a three-dimensional mesh model in order to construct a three-dimensional wound surface that allows accurate computation of surface area. In some implementations, when the wound area is relatively small and could be fully captured in a single acquisition, the processor 10 may generate the mesh directly from a single point cloud dataset using, for example, a ball-pivoting algorithm, a Poisson surface reconstruction algorithm, or a Delaunay triangulation algorithm. In another implementations, when a large area wound requires image capture from multiple viewpoints, the processor 10 may first perform feature detection (e.g., using a SIFT or ORB algorithm) on the multiple point cloud datasets, then execute a fine alignment process using an iterative closest point (ICP) algorithm, and finally fuse the multiple point clouds into a complete mesh model using a technique, such as a truncated signed distance function (TSDF).

Finally, when calculating the percentage of total body surface area affected by the burn injury (Total Body Surface Area of Burn Injury, % TBSA), the processor 10 may first compute the actual burned surface area (Burned BSA) from the three-dimensional mesh model generated in the foregoing steps. The processor 10 may then reference a digital human body model based on a Lund-Browder chart, where the digital model predefines the standard proportional of each body region to the total body surface area. By comparing the computed wound area with the corresponding region in the standard model, the processor 10 may estimate a burn area percentage (% TBSA) that more closely conforms to clinical practice (including applicability across different patient age groups). However, the present disclosure imposes no particular limitation on the method used to calculate the burn area percentage relative to the total body surface area.

In some implementations, the processor 10 may analyze the temperature of the wound region based on the wound region mask and the thermal image and the visible light image of the aligned images.

Specifically, the ambient temperature may affect assessment of wound temperature, the thermal image may be input into a temperature calibration model to ensure that thermal image obtained under varying environmental conditions correctly reflect the wound temperature. In some implementations, the burn injury assessment system may input the thermal image into the temperature calibration model to obtain a corrected thermal image. The corrected thermal image may then be aligned with the visible light image such that each pixel in the corrected thermal image corresponds to a respective pixel location in the visible light image. Finally, the processor 10 may analyze the temperature of the wound region based on the wound region mask and the corrected thermal image and visible light image of the aligned image.

Please referring to FIG. 3, in step S309, inputting the wound region area, the plurality of physiological parameters, and the injury time parameter into a burn classification model to obtain a burn index of the wound.

In some implementations, the processor 10 may construct a feature vector based on the wound area, the quantitative values of the physiological parameters, and the injury time parameter obtained in step S307. The processor 10 may then input the feature vector into a burn classification model that has been previously trained. The physiological parameters may include, but are not limited to, values representing wound depth, water content, blood oxygen concentration, and temperature of the wound. The burn classification model is configured to receive the feature vector and output a Burn Index having a grading range from first degree to fourth degree, where the second degree may be further subdivided into superficial second degree and deep second degree. Specifically, in some implementations, the Burn Index may be expressed as first degree, superficial second degree, deep second degree, third degree, and fourth degree. In some implementations, the Burn Index may be further expressed, based on the injury time parameter, as first degree, superficial second degree, deep second degree, third degree, or fourth degree in an acute phase, subacute phase, or repair phase. The Burn Index serves as a quantitative indicator that more precisely and comprehensively represents the severity or grade of the burn injury, thereby providing a reference and recommendation for medical personnel in subsequent clinical care.

In some implementations, the method proposed according to the present disclosure could further calculate the wound area, multiple physiological parameters, and the burn index of the wound region, via a formula, and obtain a wound healing score, where the wound healing score is used to assess the future healing status of the current wound. More specifically, the processor may calculate the wound healing score, based on the burn index, wound area, water content, and blood oxygen concentration values, using the formula. The aforementioned formula is shown as Formula (1), where k represents the burn index, ωₖ represents a weight corresponding to each burn index, and Aₖ represents a percentage of the total body surface area (TBSA) occupied by a wound region having burn index k. In addition, i represents the parameter item number, ωᵢ represents the weight corresponding to the i-th parameter, ω₆ represents an area weight, P_{i(b}_{ₖ}₎ represents a value of the i-th physiological parameter for the wound region having burn index k, and Pᵢ₍ₛ₎ represents a value of the i-th physiological parameter corresponding to healthy skin. The physiological parameters Pᵢ may include blood oxygen saturation (StO₂), deoxygenated hemoglobin (Hb), oxygenated hemoglobin (HbO₂), water content, and wound temperature.

Additionally, since an individual's burn wounds may be distributed across multiple body regions, the overall burn injury region may include: multiple wound regions having different burn index classifications, multiple wound regions having the same burn index classification, or multiple wound regions having partially different burn index classifications. Accordingly, a sum of all Aₖ values corresponds to the total wound area. When calculating the wound healing score, different weights are assigned according to different burn indexes in the wound, where a higher burn index is given a greater weight. Next, the weight corresponding to the burn index is then multiplied by comprehensive information corresponding to the wound associated with that burn index. The comprehensive information includes physiological parameters of the wound and a wound area, each multiplied by their corresponding weights. In calculating the physiological parameters of the wound, the burn region is further compared with a healthy skin region. The weights corresponding to the physiological parameters and the wound area may reference weights of different parameters in a pre-trained burn classification model. The wound healing score ranges between 0 and 10, where 10 indicates poor healing and 0 indicates near-complete healing.

$${\sum }_{k} {ω}_{k} \times ( {\sum }_{i = 1}^{5} {ω}_{i} \frac{\vert {P}_{i ( {b}_{k} )} - {P}_{i ( s )} \vert}{{P}_{i ( s )}} + {ω}_{6} \times {A}_{k} ( \% ) ) \qquad Formula ( 1 )$$

In some implementations, a healing score of 0 is assigned when no burn injury is present, and physiological information from a healthy skin region of the same subject may be used as a reference baseline to reduce inter-subject variability and minimize result deviation. All weight values are real numbers greater than 0. For example, a dataset may indicate that a wound exhibits a healing score of 10 on the first day of imaging, a score of 8 after one week, and a score of 6.7 after an additional week, indicating a good wound healing status.

In some implementations, the training dataset for the burn classification model may be obtained from a collection of multiple burn cases, and the injury time of each burn case may be recorded. For each burn case, wound region images may be captured using a multispectral image sensor, a depth image sensor, and a thermal image sensor in the burn injury assessment system. The image segmentation model may then be used to segment the wound region from the images and generate a corresponding mask. By analyzing visible light images, depth images, multispectral images, and thermal images corresponding to the mask, the burn injury assessment system may compute a set of quantitative physiological parameters and a wound area. The physiological parameters may include, but are not limited to, wound depth, blood oxygen concentration values, water content, and wound temperature distribution.

In some implementations, during the labeling stage of the training data, burn cases collected and stored in a cloud database may be downloaded, in which each burn case includes a corresponding injury time parameter (i.e., a time interval from occurrence of the burn to image acquisition, typically measured in hours). For each burn case, a processor in the burn injury assessment system may analyze and compute physiological parameters and wound area based on the acquired images, where the physiological parameters include wound depth, blood oxygen concentration values, water content, and wound temperature distribution. The computed physiological parameters, wound area, and injury time parameter may be automatically transmitted to a labeling device (not shown in figures) in the burn injury assessment system via a wireless communication unit. The labeling device may output a reference label (ground-truth label) corresponding to the burn wound, such as a burn index (e.g., first degree, superficial second degree, deep second degree, third degree, or fourth degree). In some implementations, the labeling device may include an automated labeling device. In some implementations, the labeling device may include a semi-automated labeling device that enables a user (e.g., medical personnel) to review or correct the classification based on the physiological parameters, wound area, and injury time parameter. Accordingly, each training data may include: (1) an input feature vector composed of quantitative values corresponding to wound area, injury time, and the physiological parameters; and (2) a burn index as an output label.

In some implementations, during the labeling stage of the training data, burn cases (e.g., murine subjects) collected and stored in a cloud database may be downloaded, in which each burn case includes a corresponding injury time parameter (i.e., a time interval from occurrence of the burn to image acquisition, typically measured in hours). For each burn case (e.g., murine subjects), a processor in the burn injury assessment system may analyze and compute physiological parameters and wound area based on the acquired images, where the physiological parameters include wound depth, blood oxygen concentration values, water content, and wound temperature distribution. In some implementations, the parameters of the Beer-Lambert model may be adjusted to convert a model developed based on murine imaging and tissue analysis to a human-applicable model. Specifically, reflectance data of human skin at different spectra may be referenced from Liu, Xinyu et al., “Wavelength selection for real-time detection of human stress based on StO₂,” while reflectance data of murine skin at different spectra may be referenced from Sabino C P et al., “The optical properties of mouse skin in the visible and near infrared spectral regions.” The human reflectance may then be substituted into the reflectance component of the Beer-Lambert formula for human skin (e.g., as referenced in Tsai, Hsin-Yi et al., “A noncontact skin oxygen-saturation imaging system for measuring human tissue oxygen saturation”), such that a Beer-Lambert formula originally used to compute murine physiological parameters may be transformed to a Beer-Lambert formula for calculating human physiological parameters. The computed physiological parameters, wound area, and injury time parameter may be automatically transmitted to a labeling device (not shown in figures) in the burn injury assessment system via a wireless communication unit. The labeling device may output a reference label (ground-truth label) corresponding to the burn wound, such as a burn index (e.g., first degree, superficial second degree, deep second degree, third degree, or fourth degree). Accordingly, each training data may include: (1) an input feature vector composed of quantitative values corresponding to wound area, injury time, and the physiological parameters; and (2) a burn index as an output label.

In some implementations, the burn classification model may include a multilayer perceptron (MLP), a convolutional neural network (CNN), a support vector machine (SVM), a k-nearest neighbors (KNN) algorithm, or a recurrent neural network (RNN). In preferred implementations, the burn classification model is a multilayer perceptron (MLP).

To train the burn classification model, the dataset may first be randomly divided, such that 80% of the data is used as a training set and 20% is used as a test set. The training process may employ a supervised learning approach, in which a cross-entropy loss function is used to quantify the difference between the model's predictions and the ground truth labels, and the model weights are updated iteratively. The training procedure may be performed for 200 epochs and may further incorporate cross-validation to evaluate the generalization capability of the burn classification model, thereby ensuring that the final trained burn classification model could generate accurate and stable predictions for the input data.

After training is completed, the burn classification model may be deployed within the burn injury assessment system. The burn classification model is capable of receiving real-time physiological parameters that are computed from the images and generating an objective, reliable, and accurate burn classification result to assist clinical diagnosis. In addition, each image that is processed by the burn injury assessment method disclosed herein requires approximately 0.009±0.012 milliseconds (ms).

Accordingly, the burn injury assessment system and burn injury assessment method of the present disclosure could effectively evaluate the Burn Index of a burn case by analyzing wound region image data, thereby enabling early planning of subsequent medical treatment and reducing the risk of delayed care.

In some implementations, Table 1 illustrates the effect of removing an individual feature on the accuracy of the training of the burn classification model. Specifically, the present disclosure systematically removes one feature at a time to evaluate the model's accuracy, thereby demonstrating the key influence of each input parameter on the prediction results. Unlike conventional single feature evaluation approaches, the present disclosure confirms that the selection and combination of these physiological parameters are derived and optimized through experimentation rather than by arbitrary choice, and are critical to achieving high accuracy.

In the study, the burn classification model is first trained and evaluated using the complete feature set to establish a baseline performance. For example, when all features are included, the burn classification model achieves an accuracy of approximately 83.70%+1.28%. Subsequently, each feature is systematically removed from the input dataset, and the model performance is re-evaluated. A larger decrease in accuracy following the removal of a particular feature indicates a greater importance of that feature in the model's decision-making process.

In some implementations, removal of deoxygenated hemoglobin (Hb) or the injury time parameter results in the most significant reductions in accuracy, with both causing approximately an 18.52% decrease. Removing other features also results in substantial accuracy drops; for example, removal of tissue oxygen saturation (StO₂) results in a decrease of approximately 17.04%, removal of water content results in a decrease of approximately 16.30%, removal of wound temperature results in a decrease of approximately 15.56%, removal of wound area results in a decrease of approximately 10.37%, and removal of oxyhemoglobin (HbO₂) results in a decrease of approximately 5.93%.

Such analysis demonstrates that parameters, such as deoxygenated hemoglobin (Hb) and injury time, are critical to the performance of the burn classification model. The specific weights and interactions of these parameters learned by the neural network during a training process are indispensable for achieving high accuracy in burn classification.

In some implementations, when computing a wound healing score, the weights corresponding to the physiological parameters and wound area may reference the parameter weights learned by the pretrained burn classification model. Specifically, changes in deoxygenated hemoglobin (Hb) serve as the primary factor influencing the wound healing score. A decrease in deoxygenated hemoglobin (Hb) may indicate that the wound has begun to demonstrate signs of healing. Conversely, an increase in deoxygenated hemoglobin (Hb) may signal deterioration of the wound condition and the need for re-evaluation of the treatment plan. Furthermore, if the current wound healing score is higher than the previous score, the wound may be worsening and require further clinical assessment. If the current score is lower than the previous score, the wound may be exhibiting signs of healing.

TABLE 1

| | Burn classification model | Accuracy Change |
|---|---|---|
| Removed Feature | accuracy (Mean ± Std) | (vs. Baseline) |
| Baseline (all features) | 83.70% ± 1.28% | 0.00% |
| Oxyhemoglobin (HbO₂) | 77.78% ± 2.22% | −5.93% |
| Wound Area (Area) | 73.33% ± 12.37% | −10.37% |
| Temperature | 68.15% ± 7.14% | −15.56% |
| Water Content | 67.41% ± 12.24% | −16.30% |
| Tissue Oxygen Saturation | 66.67% ± 12.37% | −17.04% |
| (StO₂) | | |
| Injury Time | 65.19% ± 20.16% | −18.52% |
| Deoxygenated Hemoglobin | 65.19% ± 12.64% | −18.52% |
| (Hb) | | |

In some implementations, Table 2 illustrates the results obtained by using a same image segmentation model in combination with different burn classification models during training.

In some implementations, an FCN model may be employed as the image segmentation model. In addition, the burn classification model may include an MLP, an SVM, or a KNN. As shown in Table 2, performance evaluations were conducted for burn classification tasks using these three different classification models. The results indicate that the model adopting the MLP achieves an average accuracy of 83.70% with a low standard deviation of ±1.28%. In contrast, the SVM-based model achieves an accuracy of 53.14±8.69%, and the KNN-based model achieves an accuracy of only 49.19±7.64%. Based on the above, the MLP classification model significantly outperforms the SVM and KNN models in terms of burn classification accuracy. Furthermore, the standard deviation of the MLP model is substantially lower than those of the SVM and KNN models. Specifically, when the same image segmentation model is used, incorporating the MLP as the burn classification model effectively enhances the accuracy and repeatability of burn classification results.

TABLE 2

| Model | Accuracy |
|---|---|
| MLP | 83.70 ± 1.28% |
| SVM | 53.14 ± 8.69% |
| KNN | 49.19 ± 7.64% |

In some implementations, Table 3 presents the accuracy of different burn classification systems. In Table 3, “Yadav” refers to the method disclosed in Yadav, D. P. et al., “Feature extraction based machine learning for human burn diagnosis from burn images,”, “Butt” refers to the method disclosed in Butt, Ateeq Ur Rehman et al., “Computer aided diagnosis (CAD) for segmentation and classification of burnt human skin,”, and “Khan” refers to the method disclosed in Khan, Fakhri Alam et al., “Computer-aided diagnosis for burnt skin images using deep convolutional neural network.”, “The study of present disclosure” refers to the burn injury assessment system proposed in some implementations of the present disclosure. The results show that, compared with the methods proposed by Yadav, Butt, and Khan, the burn injury assessment method in the study of present implementations achieves an overall accuracy of 83.70%, which is noticeably higher than the accuracy achieved by the other three methods.

TABLE 3

| | | Overall |
|---|---|---|
| Reference | Proposed Method | Accuracy (%) |
| Yadav | RGB image converted to Lab image and | 82.43 |
| | then combined with SVM classification | |
| Butt | RGB image combined with logistic | 74.86 |
| | regression classification | |
| Khan | RGB image combined with CNN | 79.4 |
| | classification | |
| The study | Aligned image combined with injury | 83.70 |
| of present | time parameter and MLP | |
| disclosure | | |

As described above, the burn injury assessment system and burn injury assessment method of the present disclosure could accurately identify the wound region image data of burn cases and could effectively and accurately evaluate a burn index of a burn case based on the injury time parameter and the information carried within the wound region image. The burn index may serve as a recommendation to assist medical personnel in planning subsequent treatment at an early stage, thereby enabling earlier evaluation of the wound condition.

The embodiments shown and described above and below are only examples. Many details are often found in the art. Therefore, many such details are neither shown nor described herein for the sake of brevity. Even though numerous characteristics and advantages of the present disclosure have been set forth in the foregoing description, together with details of the structure and function of the present disclosure, the present disclosure is illustrative only, and changes may be made in the details. It will therefore be appreciated that the embodiments described above and below may be modified within the scope of the claims.

What is claimed is:

1. A burn injury assessment method, comprising:
acquiring a visible light image of a wound, a depth image of the wound, a multispectral image of the wound, a thermal image of the wound, and an injury time parameter;
inputting the visible light image into an image segmentation model to obtain a wound region and a wound region mask;
aligning the depth image, the multispectral image, the visible light image, and the thermal image to generate an aligned image;
calculating a plurality of physiological parameters of the wound region and a wound region area of the wound region based on the wound region mask and the aligned image; and
inputting the wound region area, the plurality of physiological parameters, and the injury time parameter into a burn classification model to obtain a burn index of the wound.

2. The burn injury assessment method of claim 1, wherein the image segmentation model comprises a fully convolutional network (FCN).

3. The burn injury method of claim 1, wherein the burn classification model comprises a multilayer perceptron (MLP).

4. The burn injury method of claim 1, wherein the plurality of physiological parameters comprise a wound depth, a water content, a blood oxygen concentration value, and a temperature of the wound.

5. The burn injury method of claim 1, wherein acquiring the multispectral image of the wound comprises using at least one green light source, at least one red light source, and at least one near-infrared light source.

6. The burn injury assessment method of claim 5, wherein a wavelength of the at least one green light source is between 510 nm and 560 nm, a wavelength of the at least one red light source is between 630 nm and 680 nm, and a wavelength of the at least one near-infrared light source is between 730 nm and 950 nm.

7. The burn injury assessment method of claim 1, further comprising:
inputting the thermal image into a temperature calibration model to obtain a calibrated thermal image.

8. The burn injury assessment method of claim 1, further comprising:
calculating a wound healing score based on the wound region area, the plurality of physiological parameters, and the burn index.

9. The burn injury assessment method of claim 1, further comprising:
calculating a plurality of depth values of the wound region in the depth image based on the wound region mask and the depth image;
projecting the plurality of depth values onto a three-dimensional human-body model to obtain three-dimensional information; and
converting the three-dimensional information into a two-dimensional image to calculate a ratio of the wound region area relative to a total skin surface area.

10. A burn injury assessment system, comprising:
at least one processor; and
at least one memory coupled to the at least one processor and storing at least one computer-executable instruction that, when executed by the at least one processor, cause the burn injury assessment system to execute the burn injury assessment method of claim 1.
